(19) United States
(12) Patent Application Publication
(10) Pub. No.: US 2026/0345910 A1
(43) Pub. Date: Nov. 5, 2026

(54) SYRINGE SYSTEM

(71) Applicant: DARCO International Corporation, Sunrise, FL (US)

(72) Inventor: David A. RUBEN, Sunrise, FL (US)

(21) Appl. No.: 19/467,340

(22) Filed: Feb. 2, 2026

Related U.S. Application Data
(63) Continuation of application No. PCT/US2025/011673, filed on Jan. 15, 2025.
(60) Provisional application No. 63/622,668, filed on Jan. 19, 2024.

(51) Int. Cl. A61M 5/31; A61M 5/315

(52) U.S. Cl. CPC A61M 5/3134; A61M 5/31501

(57) ABSTRACT

A syringe including a barrel having a distal end and a proximal end, the barrel defining an internal cavity extending between the distal end and the proximal end. The internal cavity has a circular cross-sectional shape. The syringe includes a locking flange extending outward from an outer surface of the barrel. The syringe further includes a plurality of ribs arranged on an outer surface of the barrel. Further, the syringe includes a tip extending from the distal end of the barrel, the tip having threads.

### CROSS REFERENCE TO RELATED APPLICATIONS

This application is a continuation of International Application No PCT/US2025/011673, filed Jan. 15, 2025, which claims priority to and the benefit of U.S. Provisional Patent Application No. 63/622,668 filed 19 Jan. 2024, the disclosures of which are hereby incorporated by reference in their entirety.

### FIELD OF THE DISCLOSURE

The present disclosure relates generally to syringes and, more particularly, to a syringe used with a powered injector for a medical procedure.

### BACKGROUND OF THE DISCLOSURE

A syringe can be used with a powered injector for use in medical procedures such as angiography, computed tomography, ultrasound, and magnetic resonance. For example, a syringe may be used on an injector for injecting fluid into the vascular system of a human or animal. Accordingly, the syringe must be handled carefully to avoid injuring the vascular system receiving an injecting fluid and to ensure fluid does not leak out of the syringe or that the syringe is damaged when installing in the powered injector. Typically, these syringes are smooth and featureless, requiring further care from a user to ensure a safe medical procedure. Additionally, these syringes typically have a hollow cavity beneath a plunger holder. This cavity presents further concern in clinical environments where the syringe is used for medical procedures, specifically with ingress from the clinical environments. Accordingly, an improved syringe is needed.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure a syringe includes a barrel having a distal end and a proximal end. The barrel defines an internal cavity extending between the distal end and the proximal end, the internal cavity having a circular cross-sectional shape. The syringe further includes a locking flange extending outward from an outer surface of the barrel, the locking flange defining an octagonal shape. The syringe further includes a plurality of ribs arranged on the outer surface of the barrel. The syringe also includes a tip extending from the distal end of the barrel, and the tip includes threads. Additionally, the syringe includes a plunger assembly slidably arranged within the internal cavity, the plunger assembly including a plunger and a plunger holder. The proximal end of the barrel may have grooves configured to received a seal to seal the internal cavity of the barrel.

In an embodiment, the plurality of ribs extend between the distal end and the proximal end. The ribs may extend onto the tip. Further, the tip may be configured to receive a Luer fitting. The barrel of the syringe may further include a conical section and a cylindrical section, the conical portion of the barrel at least partially defining a conical shape and the cylindrical portion at least partially defining a cylindrical shape. The ribs extend onto the conical shape. In some embodiments, the outer surface of the barrel defines a non-circular cross-sectional shape. Accordingly, the portions of the outer surface of the barrel may be flat. In another embodiment, the ribs may be arranged circumferentially around the outer surface of the barrel.

In yet another embodiment, the ribs may be recesses in the outer surface of the barrel. Accordingly, the recesses may extend from the distal portion to the proximal portion on the outer surface of the barrel. Additionally or alternatively, the recesses may be circumferentially arranged on the outer surface of the barrel.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is an exploded diametric front view of an example syringe assembly, according to one or more embodiments.

FIG. 2 is an exploded diametric back view of the example syringe assembly of FIG. 1.

FIG. 3 is a top view of the example syringe of FIGS. 1-2 when assembled.

FIG. 4 is a top view of another example syringe assembly, according one or more alternate embodiments.

FIG. 5 is a transparent side view of the example syringe assembly of FIGS. 1-4 when assembled.

FIGS. 6A is an upper perspective view of example seal utilizable with syringe assemblies disclosed herein, according to one or more embodiments.

FIG. 6B is a lower perspective view of the example seal of FIG. 6A.

FIG. 7 is a cross-sectional side view of a seal coupled to a syringe assembly, according to one or more embodiments.

FIG. 8A is a side view of another example syringe assembly, according to one or more embodiments.

FIG. 8B is a side view of an additional example syringe assembly, according to one or more embodiments.

FIG. 8C is a side view of yet another example syringe assembly, according to one or more embodiments.

FIG. 9A is a perspective view of another example syringe assembly, according to one or more embodiments.

FIG. 9B is a top view of the example syringe assembly of FIG. 9A, according to one or more embodiments.

FIG. 9C is a side view of the example syringe assembly of FIGS. 9A-9B, according to one or more embodiments.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to syringes and, more particularly, to a syringe used with a powered injector for a medical procedure. The syringe of the present disclosure is to be used with a powered injector and includes several elements, including at least a syringe tip, syringe barrel, plunger or plunger cover, and plunger holder. The syringe leverages features on the exterior of the syringe barrel that could run from the distal end of the syringe to the proximal end partially or entirely, meeting at the luer. These elements could encompass ridges, lines, serrations, external nubs, externally molded recesses, stippling, or any other feature that could be provided symmetrically or asymmetrically across the exterior of the syringe. These features on the exterior of the surface barrel provide a user of the syringe with an ergonomic grip. Based on the configuration of the features, the exterior of the syringe barrel can have increased friction between the user and the syringe, enhanced tactile feedback, reduced grip force, and improved comfort compared to the smooth surfaces of existing syringes. In clinical environments where safety concerns are presented during medical procedures, the features of the syringe barrel provide enhanced safety by reducing the risk of accidents caused by slipping and giving the user improved control and comfort while using the syringe.

The syringe further includes a seal, which is an element that interfaces with a distal end of the syringe barrel that can be inserted in the cavity beneath the plunger holder. The seal can be inserted with the inner diameter of the syringe barrel or interface with the outer diameter of the syringe barrel. The seal can be removed by a user without the use of specialized equipment or methods. In an example, the seal can employ tabs that are engaged by user to pry or pull the seal away to from the distal end of the syringe. The seal is placed to curtail possible ingress of particulate from the clinical environment of the syringe. The seal further covers the distal cavity of the syringe barrel and acts as a protective barrier and can further provide a visual check to the user that the syringe was verified during the manufacturing process. Accordingly, the features of the exterior of the syringe barrel and the seal of the syringe improve the safety and effectiveness of the syringe compared to existing systems. Specifically, the syringe of the present disclosure may be used with powered injectors for use in medical procedures such as angiography, computer topography, ultrasound, and magnetic resonance imaging (MRI).

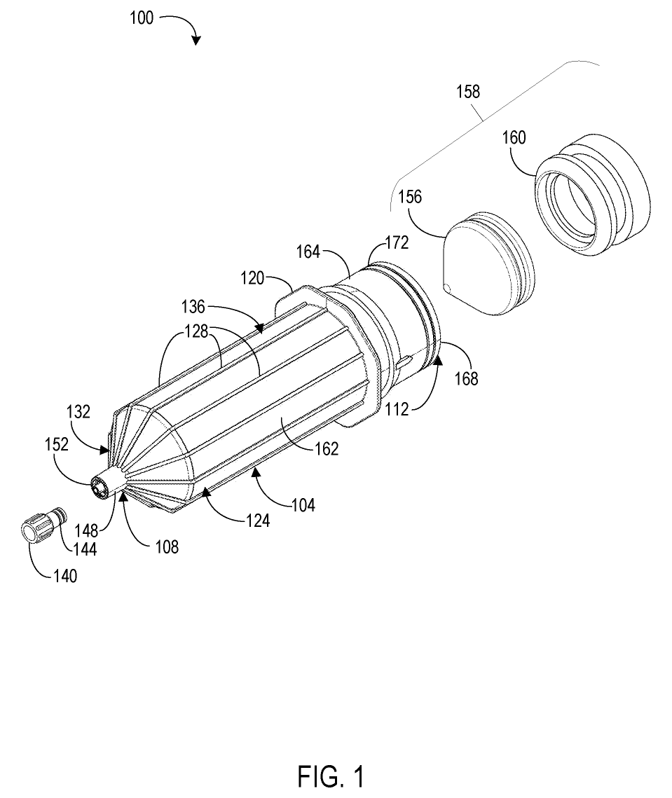

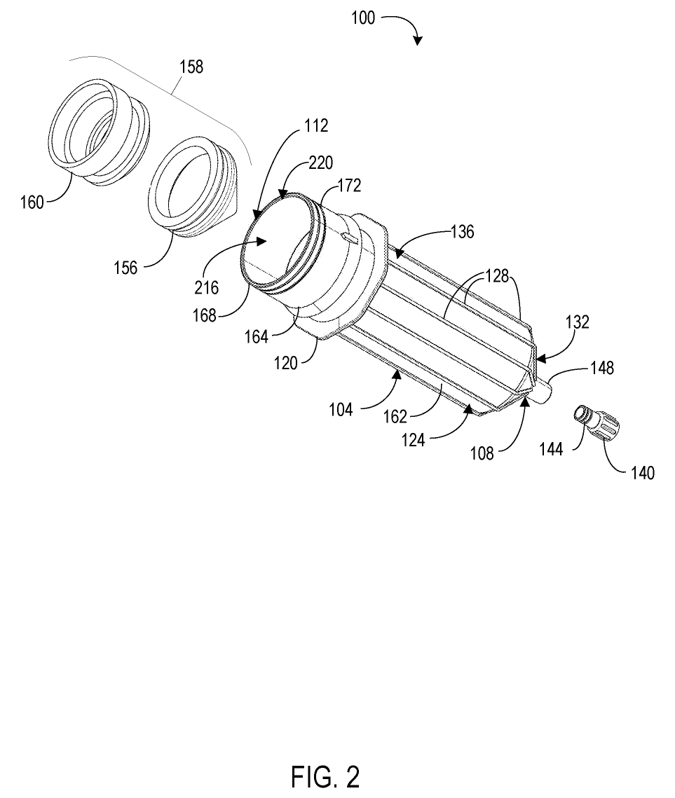

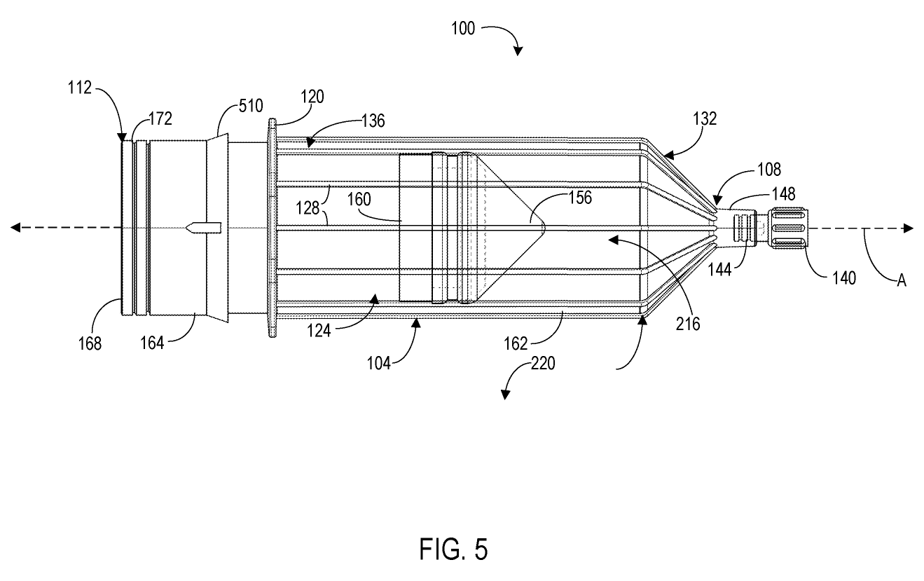

FIG. 1 illustrates a front diametric view of an example syringe assembly 100, according to one or more embodiments. The syringe assembly 100 (hereinafter, the syringe system 100 or the syringe 100) has a barrel 104 extending along a longitudinal axis A (FIG. 5) from a distal end 108 to a proximal end 112. The barrel 104 defines an internal cavity 216 (FIG. 2) extending between the distal end 108 and the proximal end 112. The internal cavity 216 of the barrel 104 may have a circular cross-sectional shape. At the proximal end 112 of the barrel 104, the syringe 100 includes a locking flange 120 (sometimes referred to as the locking flange 120) extending outward from an outer surface 124 of the barrel 104 and in a direction that is radially outward from the internal cavity 216. While FIG. 1 depicts the locking flange 120 of the example syringe 100 having an octagonal shape, the locking flange 120 may have other shapes, as discussed further herein.

On the outer surface 124 of the barrel 104, the syringe 100 includes a plurality of ribs 128. The ribs 128 are arranged/formed on the outer surface 124 to provide increased friction between a user and the syringe 100, enhanced tactile feedback, reduced grip force, and improved comfort compared to the smooth surfaces of existing syringes. The ribs 128 may extend between the distal end 108 and proximal end 112 and, in the illustrated embodiment, the ribs 128 extend between the locking flange 120 and the distal end 108. In the illustrated embodiment of FIG. 1, the ribs 128 are longitudinally extending protrusions or ridges, that extend along/parallel to the longitudinal axis A of the syringe 100, and protrude/extend radially outward from the outer surface 124 away from the longitudinal axis A.

Also in the illustrated embodiment, the barrel 104 of the syringe 100 includes a conical portion 132 at the distal end 108 and a cylindrical portion 136. The barrel 104 may also include a locking portion 164 that is configured to be received by the injection equipment and/or a seal, and the locking portion 164 may be part of the cylindrical portion 136, or the locking portion 164 may be located proximally from the cylindrical portion. In the illustrated embodiment, the locking flange 120 is located in between the locking portion 164 and the cylindrical portion 136.

As shown, the conical portion 132 extends proximally from the distal end 108 and cylindrical portion 136 extends distally (towards the distal end 108) from the proximal end 112 and/or from the locking flange 120, and the conical portion and the cylindrical portion meet each other (are joined) at a transition. Stated differently, the cylindrical portion 136 transitions into the conical portion 132, and vice versa, at the transition. The ribs 128 can extend across or along both the conical portion 132 and cylindrical portion 136 as illustrated in FIG. 1. Similar to the locking flange 120, the arrangement of ribs 128 can be differ in other examples, which will be discussed further herein. In other embodiments, for example, the ribs 128 may extend across or along just one of the conical portion 132 or the cylindrical portion 136.

The ribs 128 can generally include ridges, lines, serrations, external nubs, externally molded recesses, stippling, or any feature that could be molded, over molded, glued, or bonded chemically or physically, such as sintering. The ribs 128 can be achieved and formed by injection molding, polymer blow molding, ultrasonic welding, UV glue, two-part injection molding, over molding, or any other means that would not inhibit the function of the syringe 100. The ribs 128 can be of varying widths and lengths, and can be provided symmetrically or asymmetrically across a length (e.g., distal end 108 to proximal end 112) of the barrel 104 or perpendicular thereto. The syringe 100 and ribs 128 could be constructed from either one or a plurality of polymers, such as polypropylene, polyethylene terephthalate, polyurethane, acrylonitrile butadiene styrene, polyethylene terephthalate glycol-modified, engineered thermoplastic polyurethane, liquid crystal polymer, high density polystyrene, low density polystyrene or any number of industrial polymers widely available on the market. The ribs 128 and the barrel 104 may be formed as a unitary and monolithic structure. However, in some embodiments, prior to assembly/formation, the ribs 128 are separate structures unattached to the barrel 104, and then the ribs 128 are later attached to the barrel 104 (e.g., via welding) to define the unitary and monolithic structure of the syringe 100.

Furthermore, the syringe 100 includes a tip 148 that is integrally attached to the barrel 104 and extends distally therefrom. The tip 148 extends from the distal end 108 of the syringe 100. In the illustrated embodiment, the tip 148 is figured as a luer fitting and thus includes threads 152. Specifically, the tip 148 can have female threads 152 to form a female fitting. Thus, the tip 148 defines an apex extending from the conical portion 132. Accordingly, a connector 140 having threads 144 (e.g., male threads 144 that correspond with the female threads 152 of the tip 148) may be screwed into the tip 148 that extends from the conical portion 132. In this manner, the connector 140 can be configured as a corresponding luer fitting that is attachable to the tip 148. In the illustrated embodiment, the ribs 128 extend on the cylindrical and conical portions 132, 136, but do not extend onto the tip 148 as they instead terminate at the distal end 108 of the conical portion 132. In other embodiments, however, the ribs 128 may extend across/along the conical portion 132, the cylindrical portion 136, and the tip 148.

The connector 140 to be utilized with the syringe 100 and connectable with the tip 148 may take a plurality of forms, which may depend on the type of powered injector that the syringe 100 is utilized with, the fluid to be expelled from the syringe 100, the medical procedure, etc. The tip 148 and the connector 140 may have various configurations, including a luer slip, a 6% luer, luer slip, a luer lock, or any other configuration used for the injection of fluid or interfacing with a corresponding device for the delivery of fluid, such as a port, tube, or container of varying size, shape and construction. The connector 140 can be produced via injection molding, polymer blow molding, ultrasonic welding, UV glue, two-part injection molding, over molding, or any other means that would not inhibit the function of the syringe 100.

The internal cavity 216 of the barrel 104 may have a circular cross section, which allows a plunger assembly 158 to slidably translate through the internal cavity 216. As shown, the syringe 100 also includes the plunger assembly 158, and the plunger assembly 158 further includes a plunger 156 and a plunger holder 160. Here, the plunger 156 is conical in shape, with a circular outer diameter, similar to the conical portion 132 of the barrel 104, such that the plunger 156 may sealingly contact/engage the inner circumferential surface defined by the internal cavity 216 and thereby prevent fluid contained in internal cavity 216 from escaping out of the opening at the proximal end 112 when the plunger assembly 158 is arranged inside the internal cavity 216 of the barrel 104. Therefore, plunger 156 can travel, from the proximal end 112, to conical portion 132 at the distal end 108 of the barrel 104 (i.e., the plunger assembly 158 may travel to the transition between the conical and cylindrical portions 132, 136). The plunger holder 160 rigidly supports (i.e., provides rigidity to) the plunger 156, and facilitates placement and movement of the plunger 156 within the internal cavity of the barrel 104. The plunger holder 160 can stabilize the plunger 156, enhance control of fluid exchange, and prevent accident contamination of syringe 100 contents. The plunger holder 160 may have a generally circular outer diameter, that is slightly smaller than the inner diameter of the internal cavity 216. When assembled, the plunger 156 is at least partially arranged over the plunger holder 160, to define a single component, that may then be slidably disposed/arranged inside, with the outer circumferential edges of the plunger 156 engaging the inner circumferential surface if he internal cavity 216, to thereby form a seal between the plunger assembly 158 and the barrel 104. During use, the plunger holder 160 is engaged by a user or power injector to move/slide/translate the plunger assembly 158 towards the distal end 108 and thereby eject fluid from the syringe 100.

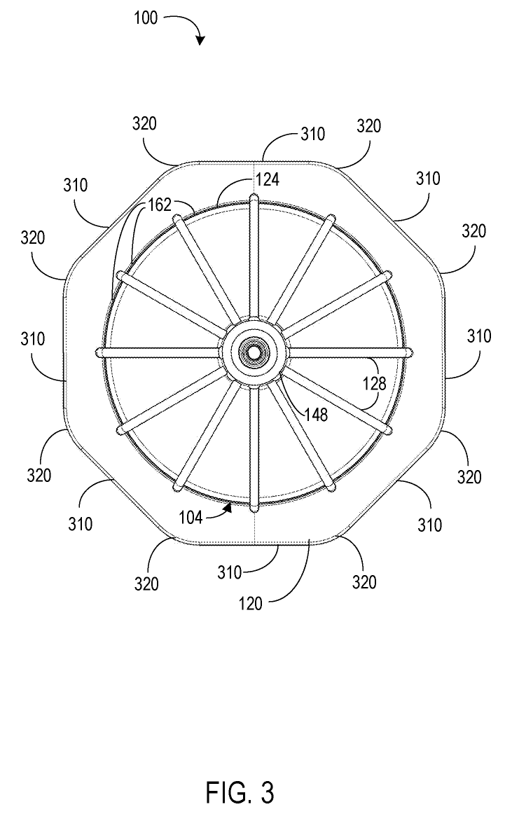

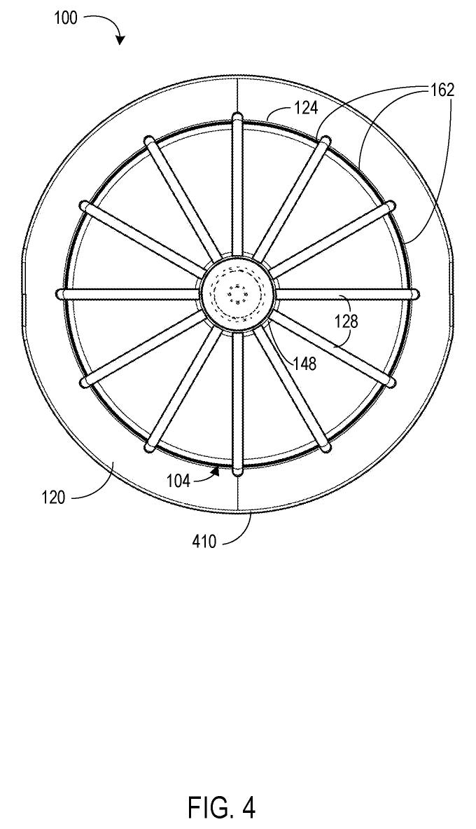

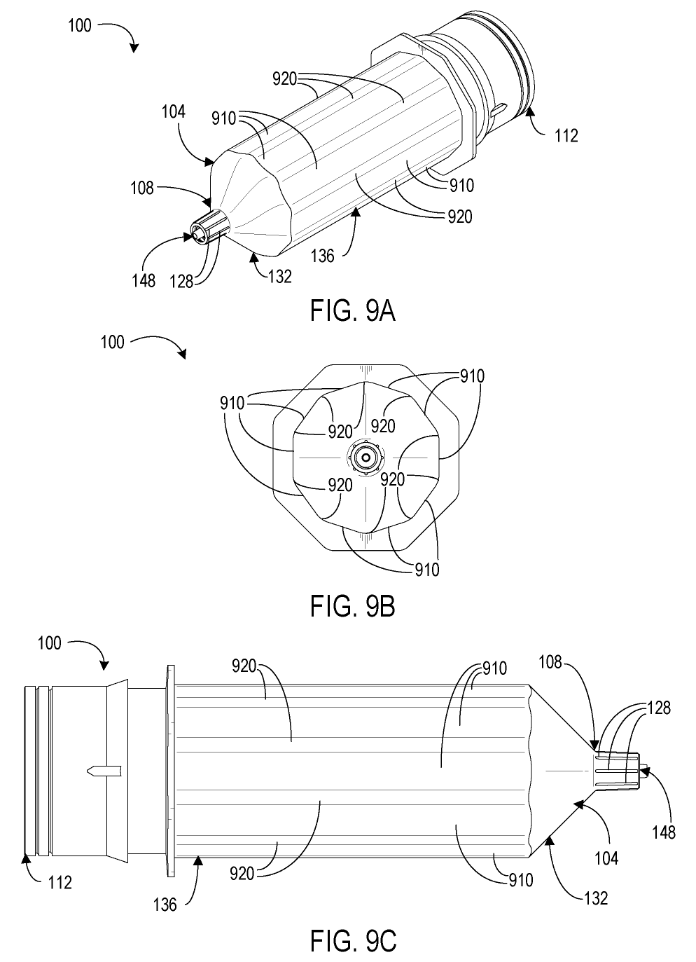

The outer surface 124 of the barrel may further include surface segments or sections 162 arranged and extending between the ribs 128 and extending longitudinally along the central axis A. In the illustrated embodiment, the surface segments or sections 162 are slightly rounded and include curvature, and when the curvature of surface segments or sections 162 are view together (e.g., in cross-section) they define a circular outer perimeter/shape that is similar to (e.g., and concentric with the circular cross-sectional shape/curvature defined by inner walls of the barrel 104 that define the internal cavity 216. In other examples, while the internal cavity 256 of the barrel 104 may have the circular cross-sectional shape to facilitate movement of the plunger assembly 158, the outer surface 124 of the barrel 104 may have a shape/curvature that differs from the surface of the internal cavity 256. For example, at least a portion of the outer surface 124 of the barrel 104 may have a non-circular shape and/or define a flat surface. For example, surface segments or sections 162 of the outer surface 124 between a given pair of the ribs 128 may be flat or non-curved (e.g., flat surfaces 910 of FIG. 9). In other examples, the surface segments or sections 162 in between ribs 128 may be triangular in shape when evaluated in cross-section, such that the overall appearance of the barrel 104 would have a star like shape when evaluated in a top view such as those shown in FIGS. 3-4. In the illustrated embodiment of FIGS. 1-2, however, the surface segments or sections 162 between the ribs 128 are curved such that together they provide the cylindrical portion 136 of the barrel 104 with a circular shaped outer perimeter, as shown in FIG. 3. In other examples, however, one or more of the surface segments or sections 162 may be flat (or define a flat surface). Thus, the outer surface 124 can have a circular cross sectional shape, when the segments are curved; or the outer surface 124 may have a polygonal cross sectional shape, when the segments are flat, where each rib 128 defines a vertex between surface segments or sections 162 of the outer surface 124. Further, the example syringe 100 of FIG. 1 illustrates each rib 128 as protruding ridges extending radially outward from the outer surface 124, thereby providing enhanced ergonomics along with the circular shape compared to smooth surfaces of existing syringes. In embodiments, one or more additional features may be provided on the surface segments or sections 162 to enhance end user’s ability to grip the syringe 100, for example, bumps, protrusions, stippling, etc.

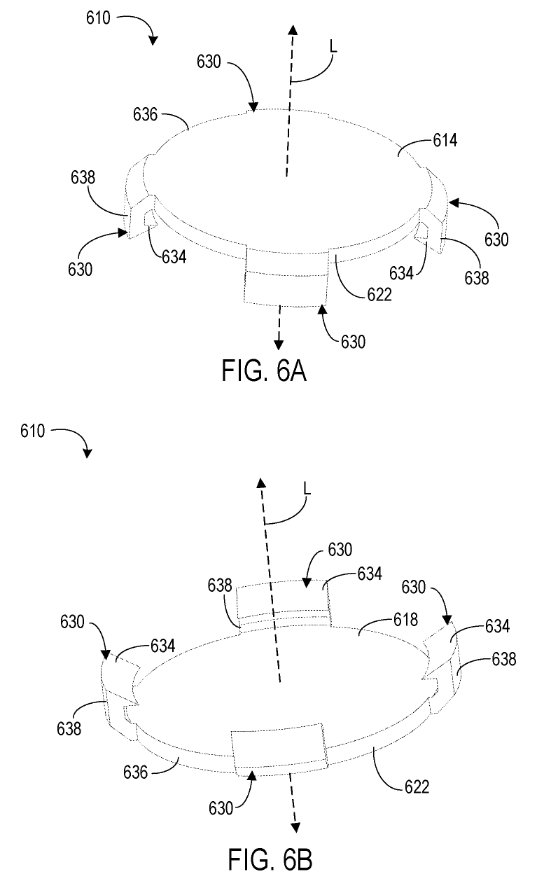

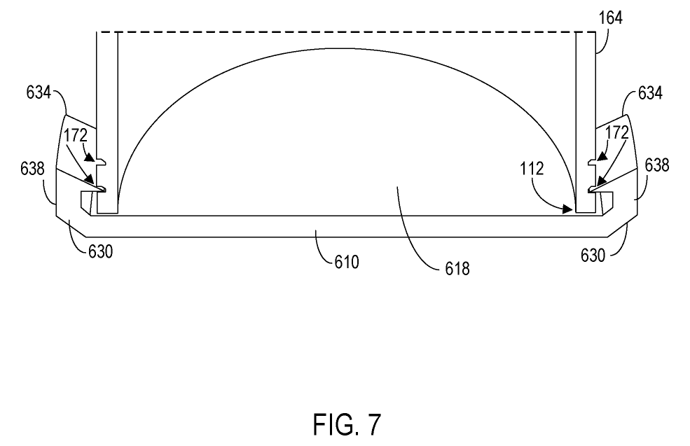

The syringe 100 further includes the locking portion 164 at the proximal end 112 of the barrel 104. Here, the locking portion 164 is generally cylindrical in shape, such that the locking portion 164 has a circular opening (FIG. 2) into which the plunger assembly 158 may be inserted and a cylindrical outer perimeter 168. The locking portion 164 further includes one or more grooves 172. In examples where the barrel 104 of the syringe 100 manufactured via molding, the one or more grooves 172 may be formed/provided to facilitate such molding process of the barrel 104, more specifically, to allow easier release of the barrel 104 during the molding process. Further, the one or more grooves 172 may a receive a seal 610 (FIGS. 6A, 6B, 7), wherein the seal 610 is configured to removably attach to the locking portion 164 as hereinafter described.

Moreover, the plunger 156 may include a supple yet structurally resilient design so that it is flexible yet conforms to a given shape, and can be constructed through natural rubber, latex-derivatives, catalyzed monomers contacts, such as neoprene, ethylene propylene diene monomer, styrene butadiene, butyl rubber, fluoro-elastomers, silicone, nitrile rubber, or any number of industrial hydrocarbon derivatives available on the market. Accordingly, the plunger 156 can be made from a flexible or semi-flexible material. Thus, the plunger 156 may, for example, be manufactured by a vulcanization compression molding process. The plunger 156 , for example, be molded into a sheet and then manually hand cut (through a cutting die) from the sheet. Once cut, the this elements may, for example, be washed in a silicone wash and then assembled onto the plunger holder 160 for insertion into a syringe 100. The plunger holder 160 may be made from various types of materials operable for rigidly maintaining the shape of the plunger 156 and utilizable to be engaged by a push rod of a power injector. The plunger 156 (and possibly the plunger holder 160) may interface with fluid, such as saline, isotonic water, contrast media solution, iodine-based contrast media either ionic or nonionic, or any application deemed necessary within the field of radiology, diagnostic imaging, or any related field. The plunger 156 extends entirely to the inner-sidewalls of the barrel 104. The plunger 156 may also have protrusions or nubs that act as a gas-seal or a gasket to ensure that while under nominal use while loaded with any of the aforementioned fluids to ensure that there is no leakage during staging, filling, injection or any stage in which fluid is loaded into the syringe 100. The syringe 100 achieves pressure to inject fluid through pneumatic force applied by the push rod of the power injector to the plunger holder 160 and/or the plunger 156, to thereby slide the plunger assembly 158. Forward, sliding motion of the plunger assembly 158 within the barrel 104 pressurizes the fluid within the syringe 100, and the pressurized fluid is injected into that patient via a syringe outlet that is in communication with the tip 148 and the connector 140.

The plunger holder interacts 160 with the plunger 156 and supports the plunger 156, so that the plunger 156 maintains engagement and a seal with the inner surface of the internal cavity 216. The plunger holder interacts 160 may be constructed from one of a plurality of polymers, such as polypropylene, polyethylene terephthalate, polyurethane, acrylonitrile butadiene styrene, polyethylene terephthalate glycol-modified, engineered thermoplastic polyurethane, liquid crystal polymer, high density polystyrene, low density polystyrene or any number of industrial polymers widely available on the market. The plunger holder 160 may be manufactured by injection molding, polymer blow molding, ultrasonic welding, UV glue, two-part injection molding or any other way to realize the design of the plunger holder 160. The plunger 156 may be affixed to the top of the plunger holder 160 through a multitude of mechanisms, most commonly through UV cured glue, friction, interfacing protrusions that recess into the plunger 156, threads, or any other method that ensures the plunger 156 be retained on the plunger holder 160 during transport, staging, filling, injection or any stage in which fluid is loaded into the syringe 100 or when the syringe 100 is otherwise used.

The locking portion 164 and internal cavity 216 best viewed with respect to FIG. 2, which illustrates a back diametric view of the example syringe 100 of FIG. 1, according to one or more embodiments. Here, the internal cavity 216 of the syringe 100 is visible, and is illustrated as having a substantially circular cross section. More specifically, the internal cavity 216 is visible through an opening 220 in the locking portion 164.

FIG. 3 illustrates a head-on view of the syringe 100, according to embodiments. As illustrated, the surface segments or sections 162 of the barrel 104, which are rounded and include a curvature in the illustrated embodiment, together generally define a substantially circular outer surface 124 of the barrel 104. The ribs 128 are also illustrated in FIG. 3 as extending to, but not onto, the tip 148. Further, FIG. 3 depicts the octagonal locking flange 120 from the top of the syringe 100. The locking flange 120 can have varying shapes, such as a polygonal shape as shown in FIGS. 1-3. That is, the locking flange 120 can have edges or sides 310 with a corresponding number of vertices 320 to form a shape consistent with desired clinical environments. That is, the locking flange 120 can various shapes according to the desired clinical environment. FIGS. 1-3 depict an example where the locking flange 120 is an octagon shape, and such octagonal shape of the locking flange 120 provides the locking flange 120 with eight (8) discrete sides 310 as best shown in FIG. 3. FIG. 4 illustrates a head-on view of the another example of the syringe 100, wherein the locking flange 120 is substantially circular in shape. That is, the locking flange 120 of the embodiment of FIG. 4 is shaped as a circle, when evaluated from the top view (FIG. 4), or from a bottom view, or when viewed in cross-section through the locking flange 120. Because the locking flange 120 is shaped as a circle in this embodiment, the locking flange 120 has substantially one side 410 and this single side 410 of the locking flange 120 is the outer circumference of the circular locking flange 120. In other examples, the locking flange 120 may have other shapes, and such other types of shapes may include polygonal shapes, oval shapes, elliptical shapes, etc.

FIG. 5 is a transparent and assembled view of the example syringe 100 of FIG. 1. Here, the tip 148 is screwed into the tip 148 of the conical portion 132 of the barrel 104. Further, the plunger 156 and plunger holder 160, are assembly and slidably inserted into the barrel 104. FIG. 5 illustrates the ability of the plunger assembly 158 to slidably translate from the proximal end 112 to the distal end 108 within the internal cavity 216 of the barrel 104, which can be effectuated by pneumatic pressure applied by an external device. Furthermore, FIG. 5 illustrates the locking portion 164 as having a stop flange 510.

Referring back to FIG. 2, the opening 220 in the barrel 104 is defined in the locking portion 164 (i.e., the locking portion 164 includes the opening 220). To prevent unwanted particulate (e.g., from the clinical environment, or particulate encountered when manufactured, transported, stored, etc.) from entering the syringe 100, the syringe can further include the seal 610. FIGS. 6A and 6B illustrate the seal 610, according to one or more embodiments. Specifically, FIG. 6A illustrates a top diametric view of the seal 610 and FIG. 6B illustrates a bottom diametric view of the seal 610, according to one or more embodiments. The seal 610 may be secured to the locking portion 164 and, more specifically, to the one or more grooves 172 defined on the locking portion 164. The seal 610 can have a substantially similar shape to the locking portion 164, and may be sized and shaped so as to obstruct and cover the opening 220, to thereby inhibit unwanted debris from entering the internal cavity 216. Here, the seal 610 has a circular shape with a size matching the opening 220 of the locking portion 164.

The seal 610 has a top surface 614 (FIG. 6A) and a bottom surface 618 (FIG. 6B), wherein the bottom surface 618 opposes (or is opposite of) the top surface 614. The seal 610 also has a peripheral side 622, forming the perimeter of the seal 610 and defines the shape (e.g., circle) and the thickness of the seal 610. The seal 610 further includes tabs 630 the protrude from the peripheral side 622 of the seal 610 and extend in a direction (from the top surface 614 towards the bottom surface 618) so as to extend beyond and past the bottom surface 618. The tabs 630 may further include a hook portion 634 and an extent portion 638. Specifically, the extent portion 638 of the tab 630 can extend away from the top surface 614 and beyond/past the bottom surface 618. The hook portion 634 can extend radially inward towards and a center line L of the seal 610 and parallel to the bottom surface 618. The seal 610, and more specifically the extent portion 638 of the tab 630, can be made from a flexible material to allow the hook portion 634 to “snap” into grooves 172 of the locking portion 164 in at least FIGS. 1-2.

The seal 610 includes a body portion 636, with the extent portions 638 attached to and extending from the body portion 636 as detailed above. The body portion 636 may be made from various types of materials. The body portion 636 is sized and shaped so as to occlude/block the opening 220 in the barrel 104, as detailed above. In some examples, the body portion 636 may be a flexible membrane, flexible film, semi-rigid membrane, semi-rigid film, rigid membrane, rigid film, or any sort of feature that would cover either partially or fully to occlude the distal end 108 of the syringe 100. The seal 610 is configured such that it may be removed from the barrel 104 by an end user without the use of specialized equipment, specialized methods, and under human power. Specifically, the tabs 630 can be engaged upon by the user to pry or pull the hook portion 634 out of engagement with the groove 172 and then pull the seal 610 away from the proximal end 112 of the syringe 100. Alternatively, a string or other feature may be attached to the seal 610 or attached to the barrel 104, and utilized to assist in leveraging the seal 610 away from the distal end 108, or any feature that would assist in providing either greater area or easier area for the user to graph or provide a mechanical advantage, while removal of the string/feature from the barrel 104 would not leave any trace of its being there and no part of the seal 610 should be left in the internal cavity 216. In other embodiments, the seal 610 may be attached to the proximal end 112 of the barrel 104 either by physical threading, interfacing with attachment points on the locking portion 164, friction fit within the internal cavity 216 or over an outer perimeter 168 of the barrel 104, interfacing locking lugs, interrupted threads, snap-on through friction, adhesive tabs/strips, or any other mechanism that would allow the seal 610 and barrel 104 to be attached together and withstand transit to the end user.

Furthermore, the seal 610 is made with a material able to withstand sterilization through one or more mechanisms of action, including but not limited to steam vapor, gamma radiation, or ethylene oxide, or a method that is consistent with medical device usage and is comprised of from either one or a plurality of polymers, such as polypropylene, polyethylene terephthalate, polyurethane, acrylonitrile butadiene styrene, polyethylene terephthalate glycol-modified, engineered thermoplastic polyurethane, liquid crystal polymer, high density polystyrene, low density polystyrene or any number of industrial polymers widely available on the market. Further, the seal 610 may be manufactured various methods, including but not limited to injection molding, polymer blow molding, ultrasonic welding, UV glue, two-part injection molding or any other way to realize the design of the seal 610.

FIG. 7 illustrates a cross-sectional view of the locking portion 164 of the syringe 100 when the seal 610 is engaged thereon, according to one or more embodiments. As illustrated in FIG. 7, the hook portions 634 of locking tabs 630 are engaged within the grooves 172 of the locking portion 164. When the seal 610 is engaged with the locking portion 164, the bottom surface 618 of the seal 610 occludes and blocks the opening 220 of the locking portion 164, thereby sealing the opening 220 closed, which in turn facilitates preventing contaminates or particulate from entering the barrel 104 of the syringe 100 and the plunger assembly 158.

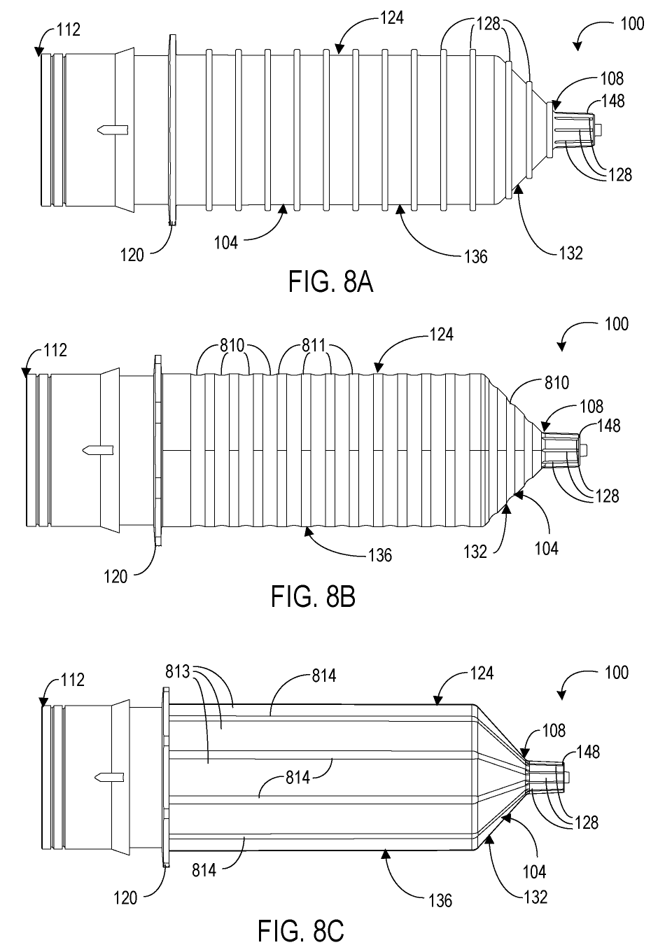

FIGS. 8A-8C illustrate alternative designs of the syringe 100, according to various alternate embodiments. The syringe barrel 104 detailed above with respect to FIGS. 1-7 may be configured as detailed with respect to FIGS. 8A-8C. Stated differently, the syringe barrels 104 described with reference to FIGS. 8A-C can be the syringe barrel 104 employed with the example syringes 100 detailed herein. As previously noted, differing arrangements of ribs 128 can be employed to offer varying ergonomic grips, such as the ribs 128 extending from the distal end 108 to the locking flange 120 in FIGS. 1-3. In FIG. 8A, the ribs 128 are arranged circumferentially around the outer surface 124 of the barrel 104, so that each of the ribs 128 resembles a ring that protrudes from the outer surface 124. By arranging the ribs 128 circumferentially around the outer surface 124 in this manner, the ribs 128 can enhance the strength and rigidity of the barrel 104 to prevent deformation under pressure. In FIG. 8A, the ribs 128 protrude from the outer surface 124 of the barrel 104. Also in FIG. 8A, the locking flange 120 is shaped like an octagon, but in other embodiments, the locking flange 120 may have other shapes, such as a circular shape or other polygonal shape.

In FIG. 8B, the outer surface 124 of the barrel 104 has recesses 810, instead of protruding ribs 128. In some examples, the recesses 810 define annular recesses, with relatively raised portions 811 being defined in between neighboring recesses 810, and with the relatively raised portions 811 effectively defining “ribs” that are ring shaped and extending around the barrel 104 as shown. The recesses 810, together with relatively raised portions 811 therebetween, create friction on the outer surface 124 to improve ergonomics of the barrel 104 similar to the ribs 128. Thus, FIG. 8B depicts the outer surface 124 of the barrel 104 having recesses 810 that are arranged circumferentially around the outer surface 124 of the barrel 104 to thereby define the raised portions 811 (or ribs) therebetween. Also in FIG. 8B, the locking flange 120 is shaped like an octagon, but in other embodiments, the locking flange 120 may have other shapes, such as a circular shape or other polygonal shape. In even other examples, the outer surface 124 of the barrel 104 can include a combination of both ribs 128 and recesses 810 and, in such embodiments, the ribs 128 may extend longitudinally as shown in FIG. 1 or the ribs 128 may be arranged on the raised portions 811 and define ring shaped protrusions that are oriented similar to the raised portions 811 so as to define rings. Again, both ribs 128 and recesses 810 can be at the cylindrical portion 136 and the conical portion 132.

In another example, FIG. 8C illustrates an embodiment where there barrel 104 includes a plurality of faceted flat surfaces 813 with curved/rounded transitions 814 arranged between each of the flat surfaces 813. In this manner, rather than the flat surfaces 813 joining each other at sharp joints, they gently transition into each other via a curvature defined by the rounded transitions 814. The flat surfaces 813, and the curved transitions 814 arranged therebetween, all extend longitudinally from the distal end 108 to the locking flange 120 of the barrel 104. Here, for example, the flat surfaces 813 and the curved transitions 814 both extend onto the conical portion 132 of the barrel 104. However, in other embodiments, either or both of the flat surfaces 813 and the curved transitions 814 do not extend onto the conical portion 132 of the barrel 104. Also in FIG. 8C, the locking flange 120 is shaped like an octagon, but in other embodiments, the locking flange 120 may have other shapes, such as a circular shape or other polygonal shape. Also, in the embodiments shown in FIGS. 8A-8C, the syringes 100 include ribs 128 that extend longitudinally (parallel to the longitudinal axis A) along the tip 148.

FIGS. 9A-9C illustrate another example of syringe barrel 104 having features on the outer surface 124 to provide an ergonomic syringe 100. Here, the outer surface 124 of the barrel 104 does not have ribs 128 as detailed above with respect to FIG. 1. Instead, the outer surface 124 of the barrel 104 includes faceted flat surfaces 910 connected by rounded corners 920, similar to the embodiment depicted in FIG. 8C. In this embodiment, the conical portion 132 does not have ribs 128. Further in this embodiment, the conical portion 132 does not include the flat surfaces 910 or any other features (such as the ribs 128), but the tip 148 includes longitudinal ribs 128 similar as described with reference to FIGS. 8A-8C. Instead, in this embodiment the conical portion 132 is purely conical in shape, but does not include any ribs 128 (like the tip 148) nor a plurality of faceted flat surfaces 910 and rounded corners 920 (like the outer surface 124 of the barrel 104). In some alternate examples, however, the tip 148 does not include ribs 128. As best depicted in the top view of the syringe 100 in FIG. 9B, the outer surface 124 of the barrel 104 in the illustrated example includes ten faceted flat surfaces 910. Similarly, the outer surface 124 of the barrel 104 includes ten rounded corners 920 in between neighboring flat surfaces 910. While the example syringe barrel 104 of FIGS. 9A-9C has ten faceted flat surfaces 910 and corners 920, it is to be appreciated the syringe barrel 104 is not limited to ten and it may include more or less than 10 faceted flat surfaces 910 in other embodiments. That is, the example syringe barrel 104 can have less than or more than ten faceted flat surfaces 910 and corners 920. Furthermore, the syringe barrel 104 can have a combination of ribs 128, recesses 810, faceted flat surfaces 910 and corners 920.

The embodiments of the syringes 100 described in the present disclosure may be used with powered injectors, for example, powered injectors for use in medical procedures such as angiography, computed tomography, ultrasound and magnetic resonance imaging (MRI). The syringes 100 detailed herein are operable for injecting fluid into the human being or an animal, such as the being’s vascular system. While conventions syringes are smooth and featureless, the syringes 100 of the present disclosure have various arrangements of ribs 128 and other features that improve the ergonomics and usability of said syringes 100. Typically, syringes have a hollow cavity beneath the plunger holder 160, and within the industry there are concerns of safety specifically with ingress of particulate from the clinical environment (or elsewhere) in which the syringes are used. To remedy this concern, the embodiments of the syringes 100 disclosed herein may include the seal 610 which is operable to cover the opening 220 of the syringe barrel 104 and act as a protective barrier inhibiting unwanted particulate from entering the barrel 104, and the grooves 172 could double as a visual check to the end-user that the syringe 100 was verified during the manufacturing process and is in a sterile condition ready for use by the clinician. Specifically, the grooves 172 can be employed both to fix/couple/attach the seal 610 to the barrel 104 and provide a visual safety check.

### Embodiments disclosed herein include

A. A syringe, comprising: a barrel having a distal end and a proximal end, the barrel defining an internal cavity extending between the distal end and the proximal end, the internal cavity having a circular cross-sectional shape; a locking flange extending outward from an outer surface of the barrel, the locking flange defining an octagonal shape; and a plurality of ribs arranged on the outer surface of the barrel.

Each of embodiments A through C may have one or more of the following additional elements in any combination: Element 1: the plurality of ribs extend between the distal end and the proximal end. Element 2: a tip extending from the distal end of the barrel, wherein the tip includes threads. Element 3: the plurality of ribs extend between the distal end and the proximal end, and the plurality of ribs extend onto the tip. Element 4: the tip is configured to receive a Luer fitting. Element 5: the barrel further comprises a conical portion and a cylindrical portion, the conical portion of the barrel at least partially defining a conical shape and the cylindrical portion at least partially defining a cylindrical shape. Element 6: the ribs extend onto the conical shape. Element 7: the outer surface of the barrel defines a non-circular cross-sectional shape.

Element 8: a portion of the outer surface in between the ribs is flat. Element 9: a plunger assembly slidably arranged within the internal cavity of the barrel, wherein the plunger assembly comprises a plunger and a holder. Element 10: a seal. Element 11: grooves at the proximal end of the barrel configured to receive the seal to secure the internal cavity. Element 12: the plurality of ribs are arranged circumferentially around the outer surface of the barrel. Element 13: the plurality of ribs protrude from the outer surface of the barrel. Element 14: the plurality of ribs are annular recesses in the outer surface of the barrel.

By way of non-limiting example, exemplary combinations applicable to A include: Element 1; Element 2; Element 3; Element 3 with Element 2; Element 4 with Element 2; Element 5; Element 6 with Element 5; Element 7; Element 8 with Element 7; Element 9; Element 10 with Element 9; Element 11 with Element 10; Element 12; Element 13 with Element 12; and Element 14 with Element 13.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A syringe, comprising:
a barrel having a distal end and a proximal end, the barrel defining an internal cavity extending between the distal end and the proximal end, the internal cavity having a circular cross-sectional shape;
a locking flange extending outward at the proximal end from an outer surface of the barrel; and
a plurality of ribs arranged on the outer surface of the barrel.

2. The syringe of claim 1, wherein the plurality of ribs are equally spaced around a circumference of the barrel and extend from the proximal end of the barrel at least partially along the barrel toward the distal end of the barrel.

3. The syringe of claim 2, wherein the plurality of ribs extend between the locking flange and the distal end.

4. The syringe of claim 1, further comprising a tip extending from the distal end of the barrel, wherein the tip includes threads.

5. The syringe of claim 4, wherein the plurality of ribs extend between the locking flange and the distal end, and the plurality of ribs further extend onto the tip.

6. The syringe of claim 4, wherein the tip is configured as a Luer fitting.

7. The syringe of claim 1, wherein the barrel further comprises a conical portion and a cylindrical portion, the conical portion of the barrel at least partially defining a conical shape and the cylindrical portion at least partially defining a cylindrical shape.

8. The syringe of claim 7, wherein the ribs extend onto the conical shape.

9. The syringe of claim 1, wherein the outer surface of the barrel defines a non-circular cross-sectional shape.

10. The syringe of claim 9, wherein a portion of the outer surface in between the ribs is flat.

11. The syringe of claim 1, further comprising a plunger assembly slidably arranged within the internal cavity of the barrel, wherein the plunger assembly comprises a plunger and a holder.

12. The syringe of claim 1, further comprising a seal removably attachable to the barrel.

13. The syringe of claim 12, wherein the barrel further comprises grooves configured to receive the seal to secure the internal cavity.

14. The syringe of claim 1, wherein the plurality of ribs are arranged circumferentially around the outer surface of the barrel.

15. The syringe of claim 14, wherein the plurality of ribs protrude from the outer surface of the barrel.

16. The syringe of claim 14, wherein the plurality of ribs are annular recesses in the outer surface of the barrel.

17. The syringe of claim 1, the locking flange defining an octagonal shape.

18. The syringe of claim 1, the locking flange defining a circular shape.

19. A syringe, comprising:
a barrel having a distal end and a proximal end, the barrel defining an internal cavity extending between the distal end and the proximal end, the internal cavity having a circular cross-sectional shape;
a plunger assembly slidably arranged within the internal cavity of the barrel;
a tip extending from the distal end of the barrel, wherein the tip is configured as a Luer fitting;
a locking flange extending outward at the proximal end from an outer surface of the barrel; and
a plurality of ribs arranged on the outer surface of the barrel, wherein the plurality of ribs are molded protrusions extending from the outer surface of the barrel, and wherein the plurality of ribs are equally spaced around a circumference of the barrel and extend from the proximal end of the barrel at least partially along the barrel toward the distal end of the barrel.
